(19) United States
(12) Patent Application Publication
(10) Pub. No.: US 2001/0041708 A1
(43) Pub. Date: Nov. 15, 2001

(54) Compositions for preventing cellulite in mammalian skin

(75) Inventors: Yuan-Di Chang Halvorsen, Holly Springs, NC (US); William O. Wilkison, Bahama, NC (US); Yolanda Renee Lea-Currie, Burlington, NC (US); Peter Pieraccini, Durham, NC (US); Anindita Sen, Cary, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Zen-Bio, Inc.

(21) Appl. No.: 09/784,521

(22) Filed: Feb. 15, 2001

Related U.S. Application Data
(60) Provisional application No. 60/182,443, filed on Feb. 15, 2000.

(51) Int. Cl. A61K 7/42; A61K 31/522; A61K 31/202; A61K 35/78

(52) U.S. Cl. 514/264; 424/59; 424/725; 514/560

(57) ABSTRACT

The present invention relates to a method for combating cellulite or reducing localized fatty excesses which comprises administering to a person having cellulite or localized fatty excesses a body slimming amount of a composition containing 10-trans, 12-cis conjugated linoleic acid.

### CROSS-REFERENCE PARAGRAPH

This application claims the benefit of U.S. Provisional Application No. 60/182,443, filed Feb. 15, 2000.

### FIELD OF THE INVENTION

The present invention relates to a method for combating cellulite or reducing localized fatty excesses which comprises administering to a person having cellulite or localized fatty excesses a body slimming amount of a composition containing 10-trans, 12-cis conjugated linoleic acid.

### BACKGROUND OF THE INVENTION

Cellulite is a term applied to a skin condition associated with the lumps, bumps and dimples that appear on the thighs of many women. Cellulite primarily afflicts the thighs and buttocks but may also be present on the stomach and upper arms. This condition is frequently described as “orange peel skin”, “mattress phenomena” or the “cottage cheese effect”. Cellulite afflictions are a stubborn problem causing emotional and psychological distress to many women. Although the etiology of cellulite is poorly understood, the main etiological factor appears to be local accumulation of fat in a regional compartment.

It has been proposed that the anatomical structure of subcutaneous adipose tissue is the major cause of cellulite. The histological studies of subcutaneous tissues from men and women suggest that the fat lobules are larger and more vertical in women than men. As a result, these larger, less restricted lobules can express outward against the dermis causing the bumps and dimples characteristic of cellulite. The femoral subcutaneous fat deposits in women also tend to be more lipogenic and less lipolytic than abdominal subcutaneous or visceral fat due to the difference in the distribution of alpha and beta adrenergic receptors on adipocytes in these different regions. Increased lipolysis or fat reduction of these selected subcutaneous adipose sites may lead to a reduction or the prevention of cellulite.

Among the methods for stimulating lipolysis, the most commonly known and used is that which consists in inhibiting the phosphodiesterase in order to prevent or at least limit the rate of degradation of cyclic AMP. In effect, the phosphodiesterase destroys cyclic AMP by transforming it into 5′ AMP so that it cannot function as a lipolysis activator. Topical application for the treatment of cellulite of agents capable of distributing or reducing local fat accumulation by lipolytic action thereby improving the aesthetic appearance of the skin has been used. Among the common agents for treatment of cellulite as slimming agents are xanthine analogs such as caffeine or theophylline. These agents block the antilipolytic action of adenosine, a potent endogenous inhibitor of lipolysis.

Other known methods in lipolysis stimulation are achieved by inhibiting phosphodiesterase in order to prevent or at least limit the degradation of cAMP. Xanthine based adenosine antagonists such as caffeine or the ophylline are also known to be effective phosphodiesterase inhibitors.

Other existing methods for the treatment of cellulite have been the stimulation of adenylate cyclase to increase cAMP levels (beta-adrenergic agonists) or to block the antilipolytic inactivation of adenylate cyclase (alpha-2-adrenergic antagonists). Greenway et al. (U.S. Pat. No. 4,588,724) disclose that isoproterenol, a known beta agonist (beta-adrenergic stimulator), is effective for the treatment of cellulite by stimulating lipolysis. Greenway et al. (U.S. Pat. Nos. 4,588,724 and 4,525,359) disclose that creams based on yohimbine, a known alpha-2-blocker applied to women's skin showed a decrease in thigh circumference. Soudant et al. (U.S. Pat. No. 5,194,259) disclose a Ginkgo biloba, a known alpha-2-blocker, as a lipolytic agent in combination with at least one other alpha-2-blocker in a slimming cosmetic composition.

Moreover, it has also been known to use certain oleosoluble vegetable extracts which, according to a different mechanism, can also act as a slimming agent. For instance, in U.S. Pat. No. 4,795,638 there is disclosed a thermo slimming cosmetic composition containing an oil-soluble plant extract having slimming action. Representative of these oil-soluble plant extracts are vegetable extracts including, principally, those of climbing ivy (Hedera helix), arnica (Arnica montana), rosemary (Rosmarinus officinalis N), marigold (Calendula officinalis), sage (Salvia officinalis N), ginseng (Panax ginseng), St. Johns-wart (Hypericum perforatum), ruscus (Ruscus aculeatus), meadowsweet (Filipendula ulmaria L) and orthosiphon (Ortosifon stamincus Benth), as well as mixtures of these vegetable extracts.

Accordingly, it is an object of the present invention to provide methods for reducing or preventing cellulite in mammalian skin.

It is also an object of the present invention to provide topically applied, skin compositions for reducing or preventing cellulite containing a safe and effective amount of 10-trans, 12-cis conjugated linoleic acid.

These and other objects will become readily apparent from the detailed description, which follows.

### SUMMARY OF THE INVENTION

Compositions and methods for treating and/or preventing cellulite by administering a safe and effective amount of a skin care composition is provided. The composition comprising conjugate linoleic acid (CLA) and a pharmaceutically acceptable carrier. More particularly, the composition comprises an effective amount of 10-trans, 12-cis conjugated linoleic acid, and a dermatologically acceptable carrier for the 10-trans, 12-cis conjugated linoleic acid. The compositions of the invention improve dermal appearance by decreasing or preventing cellulite.

The present invention further relates to a skin care composition comprising from about 0.1% to about 10%, by weight, 10-trans, 12-cis conjugated linoleic acid in a package for said skin care composition. The composition may be provided with information about and/or instructions on the use of 10-trans, 12-cis conjugated linoleic acid to treat cellulite.

Unless otherwise indicated, all percentages and ratios used herein are by weight of the total composition. All weight percentages, unless otherwise indicated, are on an actives weight basis. All measurements made are at approximately 25° C., unless otherwise designated. The term “safe and effective amount” as used herein means an amount of a compound or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound judgment of the skilled artisan.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. Although specific terms are employed herein, they are used in a generic and descriptive sense only and not for purposes of limitation.

Compositions and methods for controlling or reducing localized fatty execs or cellulite are provided. The compositions comprise conjugate linoleic acid (CLA) and a pharmaceutically acceptable carrier. Conjugate linoleic acid or CLA is a mixture of isomers that can be formed from 9 cis, 12 cis-octadecadienoic acid (linoleic acid) which can, theoretically, be autoxidized or alkali-isomerized into 8 conjugated geometric isomers of 9,11- and 10,12-octadecadienoic acid (9 cis, 11 cis; 9 cis, 11 trans; 9 trans, 11 cis; 9 trans, 11 trans; 10 cis, 12 cis; 10 cis, 12 trans; 10 trans, 12 cis and 10 trans, 12 trans). The role or roles of individual isomers in particular effects was not previously known because the CLA evaluated in prior studies was a mixture of 9,1 1-octadecadienoic acids and 10,12-octadecadienoic acids and other CLA isomers. It would be advantageous to clarify these aspects of CLA activity to facilitate preparing novel compositions for administering to animals to maintain a desired biological activity while reducing an undesired activity.

Animals fed standard preparations of CLA consistently gain less weight than non-CLA fed controls. This can be a commercial disadvantage, in that it is often desirable to increase weight gain and rate of gain in animals raised to be food sources. This effect of CLA can be seen in numerous papers including, for example, Wong, M. W., et al., Anticancer Research 17:987-994 (1997); Hayek, M. G., et al., J. Nutr. 129:32-38 (1999); West, D. B., et al., Am. J. Physiol. 275 (Regulatory Integrative Comp. Physiol. 44): R667-R672 (1998); Cesano, A., et al., Anticancer Research 18:1429-1434 (1998).

CLA, particularly the 10 cis, 12 trans isomer, has direct effects on adipocytes as described in the following papers: Satroy, D. L. and Smith, S. B., J. Nutr. 129:92-97 (1999); Park, Y., et al., Lipids 34:235-241 (1999).

More particularly, the compositions of the invention comprise an effective amount of 10-trans, 12-cis conjugated linoleic acid (10t, 12c-CLA). The composition may comprise the single 10t, 12c-CLA isomer or blends of CLA as long as an effective amount of 10t, 12c-CLA is provided in the composition. The 10t, 12c-CLA isomer generally is provided at a concentration of at least about 0.1%.

By “effective amount” is an amount sufficient to provide cellulite reduction or prevention. It is accordingly an object of this invention to provide a composition that can reduce or eliminate cellulite or fat build-ups. Cellulite, as noted above, results from an accumulation of fatty materials and water imprisoned in a matrix made up of more or less watertight compartments. This matrix is comprised of elements of fundamental matter and more particularly of proteoglycons that are polymeric. For oral administration, an effective amount can be achieved by administration of at least about 0.05 gm/day to 20 gm/day, generally at least bout 1 gm/day, 2 gm/day, 3 gm/day, 4 gm/day, 5 gm/day, 6 gm/day, 7 gm/day, 8 gm/day, 9 gm/day, 10 gm/day, 11 gm/day, 12 gm/day or higher as necessary. Cellulite or fatty response to the dosage can be measured and the dosage modified accordingly. It is recognized that the dose will vary depending upon weight, age, sex, severity of obesity of the patent and the like.

As discussed in more detail below, the compositions of the invention can be formulated for oral or topical administration. For oral administration, the composition is administered in a safe and effective dosage for cellulite prevention or reduction and for the treatment of obesity. Oral administration of the composition results in decreased weight gain. Generally, for topical use, the composition is presented in the form of a cream or oil for topical administration, usually in the form of a cream. Thus, the methods of the invention encompass application of the composition used for local slimming and for fighting cellulite.

The composition according to the invention was conceived for fighting conditions of external appearance and figure, such as cellulite, general or local obesity, relaxing or ptosis of the skin and excessive secretion of fat (seborrhoea), which reveal profound bodily dysfunctions. Thus, the compositions of the invention demonstrate a slimming and “rejuvenating” effects on appearance. By using the cream of the invention, good results may be obtained in terns of slimming and of reducing cellulite. That is, the composition is useful for fighting local fat and cellulite. The skin becomes toned and fortified and the user feels no need, from an aesthetic point of view, to use another cream as a supplementing thereof.

The compositions used in the present invention can comprise, consist of, or consist essentially of the essential elements and limitations of the invention described herein, as well any of the additional or optional ingredients, components, or limitations described herein.

References herein to a “patient” are intended to refer both to human subjects with a desire to treat or prevent cellulite. References herein to “animals” can be, but are not limited to, a rodent, a mammal (such as a bovine, an ovine, a caprine, a primate and a human), and an avian animal (such as a chicken, a duck, a turkey, and a quail).

Animals treated according to the invention also have a lower wet weight body fat percentage than control animals. A body fat percentage at least about 5% lower, more preferably at least about 10% lower and most preferably at least about 25% lower than control animals is observed in animals treated according to the invention.

While it has been possible to separately observe effects on feed conversion, weight gain and body fat content in an animal by administering a mixture of conjugated linoleic acid isomers to the animal, those skilled in the art have heretofore not known which of the principal CLA isomers (9c, 11t and 10t, 12c) is responsible for which effect or effects. Nor has the interaction between specific isomers, and the effect of administering specific combinations been evaluated. It has also not heretofore been known to use selected amounts or ratios of particular CLA isomers to achieve a desired result.

10t, 12c-CLA significantly reduces body fat when administered but also significantly suppresses growth and reduces the efficiency with which feed is converted to weight and the rate of weight gain.

The effects of the 10t, 12c-CLA isomer is demonstrated by the direct effect on rodent adipocytes as exemplified by using the 3T3-L1 adipocyte cell line. These effects include increasing lipolysis of triglycerides as evidenced by increased glycerol release by the cells and decreasing triglyceride content in said cells.

This new understanding permits one skilled in the art to produce compositions that comprise specific CLA isomer blends that promote a desirable effect when administered while reducing or eliminating one or more undesirable effects. The compositions of the invention may be administered orally or applied topically.

The compositions of the present invention comprise the indicated CLA isomer, but may also contain other CLA isomers as well as other fatty acids. The isomers can be extracted from natural sources or prepared using enzymatic or biological methods known to those skilled in the art. When making preparations of the invention, the source of the isomers is not critical, one should merely determine that the 10t, 12c isomers is provided in the composition at a percentage of at least about 0.1% to about 10%. It is recognized that higher concentrations can be utilized including at least about 20%, 30%, 40%, 50%, 60%, 70%, 80%, 90% and higher. The commercial CLA can be made from oils having at least 50% linoleic acid and which can contain 95% linoleic acid or more. The cost of CLA isomers increases with increasing purity. Bulk conjugated linoleic acid isomers in a significantly purified form (98%+pure) are commercially available from Matreya, Inc. (Pleasant Gap, Pa.). However, since the source of the isomer is not critical, it is economically advantageous to use the least expensive source of CLA to make preparations according to the invention.

The compositions can comprise the 10t, 12c-CLA isomer along with other CLA isomers as a free conjugated linoleic acids, although preferably the composition comprises only the 10t, 12c isomer. The isomers are heat stable and can be used as is, or dried and powdered. Some derivatives of individual CLA isomers are also commercially available from Matreya.

The free acid forms of the isomers may be prepared by isomerizing linoleic acid. Natural CLA may also be prepared from linoleic acid by the action of W .sup.12 -cis, W .sup.11 -transisomerase from a harmless microorganism such as the Rumen bacterium Butyrivibrio fibrisolvens. Harmless microorganisms in the intestinal tracts of rats and other monogastric animals may also convert linoleic acid to CLA (S. F. Chin, W. Liu, K. Albright and M. W. Pariza, 1992, FASEB J.6:Abstract #2665). No specific method for preparing a mixture of CLA isomers is described herein, since such methods are well known to those skilled in the art. Substantial amounts of individual pure isomers can also be prepared by the method of Chen, C. -A. and C. J. Sih, “Chemoenzymatic Synthesis of Conjugated Linoleic Acid,” J. Org. Chem. 63:9620 (1998), incorporated herein by reference.

In the method of the present invention for reducing cellulite as a topical agent, a safe and effective amount of prepared CLA formulations is administered to the patient. Since CLA is a natural food ingredient and it is relatively non-toxic, the amount of CLA that can be administered is not critical as long as it is enough to be effective to achieve the desired outcome noted herein.

The methods of the present invention may take several embodiments. In the preferred embodiment, the CLA is administered in a pharmaceutical or cosmetic composition containing a safe and effective dose of the CLA. A pharmaceutically or cosmetically acceptable carrier may additionally be provided.

In some embodiments, the formulations of the invention comprise a pharmaceutically acceptable carrier. By “pharmaceutically acceptable carrier” is intended a carrier that is conventionally used in the art to facilitate the storage, administration, and/or the healing effect of the therapeutic ingredients. A carrier may also reduce any undesirable side effects of the 10t, 12c-CLA. A suitable carrier should be stable, i.e., incapable of reacting with other ingredients in the formulation. It should not produce significant local or systemic adverse effects in recipients at the dosages and concentrations employed for treatment. Such carriers are generally known in the art. Suitable carriers for this invention are those conventionally used large stable macromolecules such as albumin, for example, human serum albumin, gelatin, collagen, polysaccharide, monosaccharides, polyvinyl-pyrrolidone, polylactic acid, polyglycolic acid, polymeric amino acids, fixed oils, ethyl oleate, liposomes, glucose, sucrose, lactose, mannose, dextrose, dextran, cellulose, sorbitol, polyethylene glycol (PEG), and the like. Slow-release carriers, such as hyaluronic acid, may also be suitable. See particularly Prisell et al. (1992) Int. J. Pharmaceu. 85:51-56, and U.S. Pat. No. 5,166,331. Other acceptable components in the composition include, but are not limited to, pharmaceutically acceptable agents that modify isotonicity including water, salts, sugars, polyols, amino acids, and buffers. Examples of suitable buffers include phosphate, citrate, succinate, acetate, and other organic acids or their salts and salts that modify the tonicity such as sodium chloride, sodium phosphate, sodium sulfate, potassium chloride, and can also include the buffers listed above.

The method for formulating a pharmaceutical composition is generally known in the art. A thorough discussion of formulation and selection of pharmaceutically acceptable carriers, stabilizers, and isomolytes can be found in Remington's Pharmaceutical Sciences (18^{th} ed.; Mack Pub. Co.: Eaton, Pa., 1990), herein incorporated by reference.

In the preferred embodiment of the invention, a cosmetically acceptable vehicle is comprised either of water or of a water/solvent blend. The solvent is optimally chosen from propylene glycol, ethanol, butylene glycol, and polyethylene glycols of various molecular weights.

Vehicles other than water can include liquid or solid emollients, solvents, humectants, thickeners and powders. An especially preferred nonaqueous carrier is a polydimethyl siloxane and/or a polydimethyl phenyl siloxane. Silicones of this invention may be those with viscosities ranging anywhere from about 10 to 10,000,000 centistokes at 25° C. Especially desirable are mixtures of low and high viscosity silicones. These silicones are available from the General Electric Company under trademarks Vicasil, SE and SF and from the Dow Coming Company under the 200 and 550 Series. Amounts of silicone which can be utilized in the compositions of this invention range anywhere from 5% to 95%, preferably from 25% to 90% by weight of the composition. The cosmetically acceptable vehicle will usually form from 5% to 99.9%, preferably from 25% to 80% by weight of the emulsion, and can, in the absence of other cosmetic adjuncts, form the balance of the composition.

The compositions used in the present invention also contain a dermatologically acceptable carrier. The phrase “dermatologically-acceptable carrier”, as used herein, means that the carrier is suitable for topical application to the skin, has good aesthetic properties, is compatible with the actives of the present invention and any other components, and will not cause any untoward safety or toxicity concerns. A safe and effective amount of carrier is from about 50% to about 99.99%, preferably from about 99.9% to about 80%, more preferably from about 98% to about 90%, most preferably from about 95% to 90% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3, 1981.

The carriers of the skin care compositions can comprise from about 50% to about 99% by weight of the compositions used in the present invention, preferably from about 75% to about 99%, and most preferably from about 85% to about 95%.

Preferred cosmetically and/or pharmaceutically acceptable topical carriers include hydroalcoholic systems and oil-in-water emulsions. When the carrier is a hydro-alcoholic system, the carrier can comprise from about 0% to about 99% of ethanol, isopropanol, or mixtures thereof, and from about 1% to about 99% of water. More preferred is a carrier comprising from about 5% to about 60% of ethanol, isopropanol, or mixtures thereof, and from about 40% to about 95% of water. Especially preferred is a carrier comprising from about 20% to about 50% of ethanol, isopropanol, or mixtures thereof, and from about 50% to about 80% of water. When the carrier is an oil-in-water emulsion, the carrier can include any of the common excipient ingredients for preparing these emulsions. A more detailed discussion of suitable carriers is fount in U.S. Pat. No. 5,605,894 to Blank et al., and in PCT application WO 97/39733, published Oct. 30, 1997, to Oblong et al., both herein incorporated by reference in their entirety.

The compositions used in the present invention may optionally comprise additional materials including slimming agents as well as additional actives useful in providing cellulite control. Among these agents are phosphodiesterase inhibitors (e.g., xanthine derivatives such as theophylline, caffeine, theobromine or salts thereof such as aminophylline) and certain oleosoluble vegetable extracts, including, principally, those of climbing ivy (Hedera helix), arnica (Arnica montana), rosemary (Rosmarinus officinalis N), marigold (Calendula officinalis), sage (Salvia officinalis N), ginseng (Panax ginseng), St. Johns-wart (Hypericum perforatum), ruscus (Ruscus aculeatus), meadowsweet (Filipendula ulmaria L) and orthosiphon (Ortosifon stamincus Benth), as well as mixtures of these vegetable extracts, all of which are disclosed in U.S. Pat. No. 4,795,638, herein incorporated by reference.

Also useful are herbal and/or botanical extracts such as those disclosed in U.S. Pat. Nos. 5,705,170 and 5,667,793, both of which are herein incorporated by reference. Mixtures of any of above additional materials may also be used. The compositions used in the present invention may optionally comprise additional skin actives. Non-limiting examples of such skin actives include hydroxy acids such as salicylic acid; desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; corticosteroids such as hydrocortisone, methylprednisolone, dexamethasone, triamcinolone acetconide, and desoxametasone; anesthetics such as benzocaine, dyclonine, lidocaine and tetracaine; antipruitics such as camphor, menthol, oatmeal (colloidal), pramoxine, benzyl alcohol, phenol and resorcinol; anti-oxidants/radical scavengers such as tocopherol and esters thereof; chelators; retinoids such as retinol, retinyl palmitate, retinyl acetate, retinyl propionate, and retinal; hydroxy acids such as glycolic acid; keto acids such as pyruvic acid; N-acetyl-L-cysteine and derivatives thereof; benzofuran derivatives; and skin protectants. Mixtures of any of the above mentioned skin actives may also be used. A more detailed description of these actives is found in U.S. Pat. No. 5,605,894 to Blank et al. (previously incorporated by reference). Preferred skin actives include hydroxy acids such as salicylic acid, sunscreen, antioxidants and mixtures thereof.

Other conventional skin care product additives may also be included in the compositions used in the present invention. For example, urea, guanidine, glycerol, petrolatum, mineral oil, sugar esters and polyesters, polyolefins, methyl isostearate, ethyl isostearate, cetyl ricinoleate, isononyl isononanoate, isohexadecane, lanolin, lanolin esters, cholesterol, pyrrolidone carboxylic acid/salt (PCA), trimethyl glycine (betaine), tranexamic acid, amino acids (e.g., serine, alanine), panthenol and its derivatives, collagen, hyaluronic acid, elastin, hydrolysates, primrose oil, jojoba oil, epidermal growth factor, soybean saponins, mucopolysaccharides, and mixtures thereof may be used. Other suitable additives or skin actives are discussed in further detail in PCT application WO 97/39733, published Oct. 30, 1997, to Oblong et al., herein incorporated by reference in its entirety.

The compositions used in the present invention are generally prepared by conventional methods such as are known in the art of making topical compositions. Such methods typically involve mixing of the ingredients in one or more steps to a relatively uniform state, with or without heating, cooling, application of vacuum, and the like. Non-limiting examples of the product form can be a gel, emulsion, lotion, cream, ointment, solution, liquid, etc.

The methods of the present invention are useful for especially preventing cellulite, especially in the subcutaneous, dermis and epidermis tissues of mammalian skin. The methods of the present invention involve topically applying to the skin and effective amount of the skin care composition of the present invention. The amount of the composition which is applied, the frequency of application and the period of use will vary widely depending upon the level of 10-trans, 12-cis conjugated linoleic acid and/or other components of a given composition and the degree of cellulite fading desired.

The skin care compositions used in the present invention can be chronically applied to the skin. By “chronic topical application” is meant continued topical application of the composition over an extended period during the subject's lifetime, preferably for a period of at least about one week, more preferably for a period of at least about two weeks, even more preferably for a period of at least one month, even more preferably for at least about three months, even more preferably for at least about six months, and more preferably still for at least about one year. While benefits are obtainable after various maximum periods of use (e.g., five, ten or twenty years), it is preferred that chronic application continue throughout the subject's lifetime to maintain and/or increase the benefits achieved. Typically applications would be on the order of one to four times per day over such extended periods, however application rates can be more than four times per day, especially on areas particularly prone to agglomerations of fat and water such as the thighs and buttocks.

A wide range of quantities of the compositions used in the present invention can be employed to provide a skin appearance and/or feel benefit. Quantities of the present compositions which are typically applied per application are, in mg composition/cm.sup.2 skin, from about 0.1 mg/cm.sup.2 to about 10 mg/cm.sup.2.

The method of treating cellulite is preferably practiced by applying a composition in the form of a skin lotion, cream, gel, cosmetic, or the like which is intended to be left on the skin for some aesthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours.

Another approach to ensure a continuous exposure of the skin to at least a minimum level 10-trans, 12-cis conjugated linoleic acid is to apply the compound by use of a patch. Such an approach is particularly useful for problem skin areas needing more intensive treatment. The patch can be occlusive, semi-occlusive or non-occlusive. The 10-trans, 12-cis conjugated linoleic acid composition can be contained within the patch or be applied to the skin prior to application of the patch. The patch can also include additional actives such as chemical initiators for exothermic reactions such as those described in PCT application WO 9701313 to Burkett et al. Preferably the patch is applied at night as a form of night therapy.

The preferred xanthine employed in the inventive method is caffeine and/or theophylline due to their availability and optimum efficacy. Caffeine and theophylline can be, and preferably are naturally derived, in order to keep with a “natural” character of the inventive compositions.

The xanthine is employed in the inventive method preferably in an amount of at least 0.05%, generally in the amount of from 0.05% to 20%, preferably in the amount of from 0.10% to 10%, optimally in the amount of from 0.5% to 3.0% by weight of the composition in order to maximize efficacy at optimum cost.

Another preferred ingredient employed in the inventive method is an alpha hydroxy acid. The presence of the alpha hydroxy acid facilitates the increase in the strength and firmness of dental and epidermal layers of the skin. Even more preferably, the hydroxy acid is chosen from lactic acid, glycolic acid, mandelic acid, and mixtures thereof to optimize the efficacy of compositions by increasing percutaneous absorption. In the most preferred embodiment of the invention, in order to maximize the performance of hydroxy acid, inventive compositions contain the L-form of an alpha hydroxy acid. Preferably the amount of the alpha hydroxy acid component present in the composition according to the invention is from 1.5% to 20%, more preferably from 1.5% to 15%, and most preferably from 3.0% to 12.0% by weight of the composition.

An oil or oily material may be present, together with an emulsifier to provide either a water-in-oil emulsion or an oil-in-water emulsion, depending largely on the average hydrophilic-lipophilic balance (HLB) of the emulsifier employed.

Various types of active ingredients may be employed in the method of the present invention. Actives are defined as skin benefit agents other than emollients and other than ingredients that merely improve the physical characteristics of the composition. Although not limited to this category, general examples include sunscreens, tanning agents, skin anti-wrinkling agents, anti-inflammatory agents, skin lighteners and moisturizers.

Sunscreens include those materials commonly employed to block ultraviolet light. Illustrative compounds are the derivatives of PABA, and cinnamate. For example, octyl methoxycinnamate and 2-hydroxy-4-methoxybenzophenone (also known as oxybenzone) can be used. Octyl methoxy-cinnamate and 2-hydroxy-4-methoxy benzophenone are commercially available under the trademarks, Parsol MCX and Benzophenone-3, respectively. The exact amount of sunscreen employed in the emulsions can vary depending upon the degree of protection desired from the sun's UV radiation.

Suitable anti-inflammatory compounds include but are not limited to rosmarinic acid, glycyrrizinate derivatives, alpha bisabolol, azulene and derivatives thereof, asiaticoside, sericoside, ruscogenin, escin, esculin, quercetin, rutin, betulinic acid and derivatives thereof, catechin and derivatives thereof.

Suitable vasoactive compounds include but are not limited to papaverine, yohimbine, visnadin, khellin, bebellin, nicotinate derivatives.

Surfactants, which are also sometimes designated as emulsifiers, may be incorporated into the cosmetic compositions of the present invention. Surfactants can comprise anywhere from about 0.5% to about 30%, preferably from about 1% to about 15% by weight of the total composition. Surfactants may be cationic, nonionic, anionic, or amphoteric in nature and combinations thereof may be employed.

Illustrative of the nonionic surfactants are alkoxylated compounds based upon fatty alcohols, taffy acids and sorbitan. These materials are available, for instance, from the Shell Chemical Company under the “Neodol” designation. Copolymers of polyoxypropylene-polyoxyethylene, available under the Pluronic trademark sold by the BASF Corporation, are sometimes also useful. Alkyl polyglycosides available from the Henkel Corporation similarly can be utilized for the purposes of this invention.

Anionic-type surfactants may include fatty acid soaps, sodium lauryl sulphate, sodium lauryl ether sulphate, alkyl benzene sulphonate, mono and/or dialkyl phosphates and sodium fatty acyl isethionate.

Amphoteric surfactants include such materials as dialkylamine oxide and various types of betaines (such as cocoamido propyl betaine).

Emollients are often incorporated into cosmetic compositions of the present invention. Levels of such emollients may range from about 0.5% to about 50%, preferably between about 5% and 30% by weight of the total composition. Emollients may be classified under such general chemical categories as esters, fatty acids and alcohols; polyols and hydrocarbons.

Esters may be mono- or di-esters. Acceptable examples of fatty di-esters include dibutyl adipate, diethyl sebacate, disopropyl dimerate, and dioctyl succinate. Acceptable branched chain fatty esters include 2-ethyl-hexyl myristate, isopropyl stearate and isostearyl palmitate. Acceptable tribasic acid esters include trisopropyl trilinoleate and trilauryl citrate. Acceptable straight chain fatty esters include lauryl palmitate, myristyl lactate, oleyl eurcate and stearyl oleate. Preferred esters include coco-caprylate/caprate(a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons that may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1% to 20% by weight, preferably from about 0.5% to 10% by weight of the composition. Exemplary thickeners are cross-linked polyacrylate materials available under the trademark Carbopol from the B. F. Goodrich Company. Gums may be employed such as xanthan, carrageenan, gelatin, karaya, pectin and locust bean gum. Under certain circumstances the thickening function may be accomplished by a material also serving as a silicone or emollient. For instance, silicone gums in excess of 10 centistokes and esters such as glycerol stearate have dual functionality. Cellulosic derivatives may also be employed, e.g., hydroxypropyl cellulose (Klucel HI.RTM.).

Many cosmetic compositions, especially those containing water, must be protected against the growth of potentially harmful microorganisms. Preservatives are, therefore, necessary. Suitable preservatives include alkyl esters of p-hydroxybenzoic acid, hydantoin derivatives, propionate salts, and a variety of quaternary ammonium compounds.

Particularly preferred preservatives of this invention are methyl paraben, propyl paraben, imidazolidinyl urea, sodium dehydroxyacetate and benzyl alcohol. Preservatives will usually be employed in amounts ranging from about 0.5% to 2% by weight of the composition.

Powders may be incorporated into the cosmetic composition employed in the invention. These powders include chalk, talc, Fullers earth, kaolin, starch, smectite clays, chemically modified magnesium aluminum silicate, organically modified montmorillonite clay, hydrated aluminum silicate, filmed silica, aluminum starch octenyl succinate and mixtures thereof.

Other adjunct minor components may also be incorporated into the cosmetic compositions. These ingredients may include coloring agents, opacifiers and perfumes. Amounts of these materials may range anywhere from 0.001% up to 20% by weight of the composition.

The method of the present invention is useful for reducing or preventing the appearance of cellulite, for improving the firmness and elasticity of skin and generally to enhance the quality and flexibility of skin.

The following examples will more fully illustrate the embodiments of this invention, but the invention is not limited thereto. All parts, percentages and proportions referred to herein and in the appended claims are by weight unless otherwise indicated.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLE 1

### Lipolytic Activities of Various Isomers were Evaluated

Various positional isomers of conjugated linoleic acid were either purchased or purified and tested for lipolytic activity as described below. Lipolysis measurements performed as described below.

Day 21 differentiated human adipocytes plated in 96 well plates (Falcon, etc.) generated as described (patent, pub ref) were used. The medium was removed completely and 100 μl of the tested compound resuspended in KRB added to each well. The plates were incubated at 37° C. for 3 hrs. 100 μl of KRB from each well was transferred into the corresponding well in another 96-well plate. 2 ml distilled water was added to the glycerol assay reagent (Sigma, St. Louis Mo., catalog number) and mixed gently by inversion several times. 100 μl of this reagent was added to each well of this new plate. The solutions were mixed well either by pipetting up and down several times or by using the mix function on the plate reader and incubated at room temperature for 15 minutes. The optical density of each well was measured at 540 nm and converted to glycerol concentration by use of the glycerol standard. The increase in absorbance at 540 nm was directly proportional to glycerol concentration of the sample.

TABLE 1
LIPOLYSIS MEASUREMENT OF VARIOUS ACTIVES
% CONCENTRATION

| | | FOLD INCREASE |
|---|---|---|
| SUBSTANCE | (WT./VOLUME) | OVER CONTROL |
| Control | | 1.0 |
| CLA 10, 12 | | |
| Isoproterenal | 0.00002 | 3.4 |
| Theophylline | 0.01 | 0.8 |
| | 0.05 | 1.9 |
| | 0.1 | 3.1 |
| Caffeine | 0.1 | 2.4 |
| | 0.1 | 4.2 |

Methods

Cell Culture

Preparation and maintenance of preadipocytes. The adipocyte precursor cells (preadipocytes) are isolated from subcutaneous adipose tissue as described. The plates are kept at 37° C. with 5% CO₂ until ready for use. Differentiation into adipocytes should be initiated immediately. If cells are to be maintained as preadipocytes, they should be fed with preadipocyte medium every other day. Preadipocytes are flat, phase-dark spindle-shaped cells. The cells have a similar appearance in culture to fibroblasts or smooth muscle cells. Greater than 80% of the preadipocytes will differentiate to adipocytes using differentiation medium (DM-1). The differentiation efficiency varies depending on the donor.

Preparation and maintenance of adipocytes. Preadipocytes are differentiated into adipocytes as described. The plates are kept at 37° C. with 5% CO₂ until ready for use. The adipocytes should be fed with adipocyte medium (AM-1) every 3 days. The adipocytes should remain healthy and responsive for at least three weeks. Adipocytes are rounded, lipid-filled cells. Cultured adipocytes contain multiple vesicles termed “locules”. These locules are the site of lipid storage and can be visualized by counterstaining with oil red O. The lipolytic reaction in response to isoproterenol treatment was comparable to that of isolated primary adipocytes.

Cell Differentiation

To determine the effect of compounds on human preadipocyte differentiation, the following procedure was used. To start the treatment, remove all Preadipocyte medium from a 96-well plate of preadipocytes plated at 10,000 cell/cm². Set aside six wells for addition of the positive control, negative control, and background. Add 150 μl of the positive control to each of two wells and 150 μl of the negative control to each of two wells. To the two background wells, allow the wells to dry out during the first three days of culture. Add 150 μl of the made up initiation media to the remaining wells. Then add test compound in minimal volume (less than 5 μl) to wells of interest. Incubate plate for 3 days at 37° C. and 5% CO₂. After 3 days, cells (including the background wells) should be fed with maintenance medium every 3 days for a total of 12 days (four feedings). When feeding, remove only 100 μl of medium and replenish with 100 μl new medium since adipocytes will float if all media is taken out. At the end of the culture period, cells are fixed and stained with Oil Red O. Staining is performed as follows. Remove most of the medium (120 μl/well from 96-well plates). Add 100 μl/well fixer solution (7% formaldehyde in PBS). Keep the plate at room temperature for 5 minutes. Repeat once more by exchanging 100 μl/well of fixing solution with another 100μl/well of fresh fixer solution. Fix the cells for at least 1 hour. Cells can be fixed overnight, too. Prepare Oil Red O working solution by adding the distilled water provided into the Oil Red O stock (Oil Red O (1%) in isopropanol) solution tube. Keep the working solution for 20 minutes at room temperature before filtering through the provided filter. (Protective clothing should be used to prevent staining from the dye). Remove all the fixer. Dry all wells. Add 40 μl/well Oil Red O working solution at room temperature for 10 minutes. Be very careful not to touch the sides of the wells. Pipette tips should go straight to the bottom of the wells. Remove all the Oil Red O solution. Wash 4 times with 200 μl/well dH₂O. Remove all liquid. Add 150 μl/well isopropanol. Let the plate sit at room temperature for 10 minutes. Use pipette to stir up and down several times, making sure all the Oil Red O is back in solution. Measure the optical density at 500 nm.

### EXAMPLE 2

The following is an example of a skin cream incorporating the compositions of the present invention. The compositions are formed by combining and mixing the ingredients of each column using conventional technology and then applying to the skin from about 0.5 g to about 50 g.

| Ingredient | % Weight |
|---|---|
| Glycerine | 6.933 |
| CLA 10, 12 | 10.000 |
| Permethyl 101A.sup.1 | 3.000 |
| Sepigel.sup.2 | 2.500 |
| Q2-1403.sup.3 | 2.000 |
| Isopropyl Issosterate | 1.330 |
| Arlatone 2121.sup.4 | 1.000 |
| Cetyl Alcohol CO-1695 | 0.720 |
| SEFA Cottonate.sup.5 | 0.670 |
| Tocopherol Acetate | 0.500 |
| Panthenol | 0.500 |
| Adol 62.sup.6 | 0.480 |
| Kobo Titanium Dioxide | 0.400 |
| Sodium Hydroxide 50% Aqueous | 0.0125 |
| Fiery 5.sup.7 | 0.150 |
| Disodium EDTA | 0.100 |
| Glydant Plus.sup.8 | 0.100 |
| Myrj 59.sup.9 | 0.100 |
| Emersol 132.sup.10 | 0.100 |
| Color | 0.00165 |
| Purified Water | q.s. to 100 |

### EXAMPLE 3

The following is an example of a skin cream incorporating the compositions of the present invention. The compositions are formed by combining and mixing the ingredients of each column using conventional technology and then applying to the skin from about 0.5 g to about 50 g.

| Ingredient | % Weight |
|---|---|
| Glycerine | 6.933 |
| CLA 10, 12 | 10.000 |
| Permethyl 101A.sup.1 | 4.000 |
| Q2-1403.sup.3 | 2.000 |
| Isopropyl Isostearate | 1.330 |
| Arlatone 2121.sup.4 | 1.000 |
| Cetyl alcohol CO-1695 | 0.720 |
| sefa Cottonate.sup.5 | 0.670 |
| Carbopol 945.sup.11 | 0.500 |
| Tocopherol Acetate | 0.500 |
| Panthenol | 0.500 |
| Adol 62.sup.6 | 0.480 |
| Kobo Titanium Dioxide | 0.400 |
| Sodium Hydroxide 50% Aqueous | 0.250 |
| Fiery 5.sup.7 | 0.150 |
| Disodium EDTA | 0.100 |
| Glydant Plus.sup.8 | 0.100 |
| Myrj 59.sup.9 | 0.100 |
| Emersol 132.sup.10 | 0.100 |
| Carbopol 1382.sup.12 | 0.100 |
| Color | 0.00165 |
| Purified Water | q.s. to 100 |

sup.1 Isohexadecane, Presperse Inc., South Plainfield, NJ
sup.2 Polyacrylamide(and)C 1314 Isoparaffin(and)Laureth7, Seppic Corporation, Fairfield, NJ
sup.3 dimethicone(and)dimethiconol, Dow Coming Corp., Midland, MI
sup.4 Sorbitan Monostearate and Sucrococoate, ICI Americas Inc., Wilmington, DE
sup.5 Sucrose ester of fatty acid, Procter and Gamble, Cincinnati, OH
sup.6 Stearyl alcohol, Procter and Gamble, Cincinnati, OH
sup.7 Fiery 5 n/a, Procter and Gamble, Cincinnati, OH
sup.8 DMDM Hydantoin (and) Iodopropynyl Butylcarbamate, Lonza Inc., Fairlawn, NJ
sup.9 PEGl00 Stearate, ICI Americas Inc., Wilmington, DE
sup.10 Stearic acid, Henkel Corp., Kankakee, IL
sup.11 Carbomer, BF Goodrich, Cleveland OH
sup.12 Carbomer, BF Goodrich, Cleveland OH

### EXAMPLE 4

Trans-10, Cis-12, but not Cis-9, Trans-11, Conjugated Linoleic Acid Attenuates Lipogenesis in Primary Cultures of Stromal Vascular Cells from Human Adipose Tissue

### Introduction

Conjugated linoleic acid (CLA) consists of a group of positional and geometric fatty acid isomers that are derived from linoleic acid (18:2 n-6). CLA is found in ruminant meats, pasteurized cheeses, and dairy products and therefore is a natural part of the diet. Numerous researcher groups have demonstrated antiobesity properties of a crude mixture of CLA isomers (Houseknecht et al. (1998) Biochem. Biophys. Res. Commun. 244:678-682, West et al., (1998) Am. J. Physiol. 275:R667-R672, Park et al. (1997) Lipids 32:853-858, Park et al. (1999a) Lipids 33:243-248, Park, Y., Storkson, J., Albright, K., Liu, W., and Pariza, M. (1999b) Lipids 34:235-41, Tsuboyama-Kasaoka et al. (2000) Diabetes 49:1534-1542. For example, mice, pigs, and hamsters fed low levels of CLA (<1.5%, w/w) had less body fat and more lean body mass than controls (West et al. 1998, Park et al. 1997, Park et al. 1999a-b, Delany et al. (1999) Am. J. Physiol. 276:R1172-R1179, Cook et al. (1999) Feeding Conjugated Linoleic Acid Improves Feed Efficiency and Reduces Carcass Fat in Pigs, Adipocyte Biology and Hormone Signaling Symposium, June 7-9, p.67, Ostrawska et al. (1999) J. Nutr. 129: 2037-2042, Gavino et al. (2000) J. Nutr. 130:27-29, West et al. (2000) J. Nutr. 130:2471-2477.

Several in vitro studies have shown that treatment with of a crude mixture of 20-200 uM CLA isomers lowers the lipid content of murine (pre)adipocytes (Park et al., 1997, 1999a,b; Brodie et al. 1999, Evans et al. (2000) Lipids 35. Moreover, the trans-10, cis-12 isomer of CLA was determined to be the bioactive isomer that reduced LPL activity and TG content (Park et al. 1999b; Evans et al. 2000). Brodie et al. (1999) J. Nutr. 129:602-606 demonstrated that 25-100 uM of mixed CLA isomers inhibited both proliferation and differentiation and reduced mRNA levels of PPARγ2 and aP2 in cultures of 3T3-L1 preadipocytes. In humans, the influence of CLA treatment is less clear. For example, CLA treatment (3.4-6.8 g/d) for 3 months reduced body fat mass of obese and overweight adult men and women (Blanksen et al. (2000) J. Nutr. 130:2943-2948). In contrast, Zambell et al. (2000) Lipids 35:777-782 found that CLA consumption (3g/d-mixed isomers) over 3 mo did not affect fat mass, fat-free mass, percent body fat, or body weight in human subjects. This discrepancy may be due to the type and amount of CLA isomers used along with the body weight and energy intake status of the subjects.

Whereas CLA clearly attenuates body fat in animals and reduces the TG content of murine preadipocytes, potential antiobesity properties in humans are disputable and require further examination. Thus, examining the impact of the predominant isomers of CLA found in CLA supplements (e.g., cis-9, trans-11 and trans-10, cis-12) on the differentiation of SV cells isolated from human adipose tissue would provide insight into whether or not CLA directly influenced human adiposity. Therefore, the purpose of this study was to: 1) determine which isomer(s) of CLA attenuated TG content; 2) examine whether CLA's proposed attenuation of TG content was reversible; and 3) determine whether CLA decreased the TG content of the cultures by decreasing lipogenesis and/or increasing lipolysis in primary cultures of human adipocytes. This is the first study to demonstrate that trans-10, cis-12 CLA decreases lipogenesis in cultures of human adipocytes.

### Materials and Methods

Cell Isolation and Culture Conditions.

1) Isolation and Culture of Stromal Vascular (SV) Cells from Human Adipose Tissue.

Abdominal adipose tissue (Exp. 1-5) and thigh adipose tissue (Exp. 5b) were obtained from middle-aged females with body mass indexes <30.0 during liposuction or elective surgery. Subsequently, tissue was minced and enzymatically digested for 45 min in a Krebs-Ringer buffer containing 1 mg/mL collagenase (CLS-1, Worthington Biochemical Corp, Lakewood, N.J.), 15 mg/mL bovine serum albumin (BSA), 5 mM glucose and 100 mM HEPES (pH 7.4). Digestion was carried out at a 5 mL/1 g ratio (digestion solution: tissue mass). The digesta was then filtered through 200- and 60-micron mesh and pelleted at 600×g for 5 min. The SV cells were resuspended in a RBC lysis buffer for 10 min and then filtered and recentrifuged to remove contaminating endothelial cells. Cultures of SV cells were grown in proliferation medium containing 90% DME/Ham's F-10 (1:1, v/v), 10% (v/v) fetal bovine serum (FBS), 15 mM HEPES (pH 7.4), 60 U/mL penicillin, 60 U/mL streptomycin, and 25 ug/mL fungizone. Cultures were incubated at 37° C. in a humidified O₂:CO₂ (90:10%) atmosphere. SV cells were grown to 80% confluency and then cryopreserved in liquid nitrogen in aliquots (2×10⁶ cells/mL).

2) Induction of Cell Differentiation.

Cryopreserved aliquots were subsequently thawed, seeded in T-150 flasks (e.g., 1 vial per 2 T-150 flasks), and grown in proliferation media until 80% confluent. At this time the cells were removed via trypsinization, seeded (3×10⁴/cm², except for Exp. 1) in 24-well or 96-well (Exp. 5b) Falcon dishes, and allowed to attach for 24 h in proliferation medium. Following attachment, cultures were grown in differentiation medium for the next 3 d which contained DME/Ham's F-10 (1:1,v/v), 15 mM HEPES (pH 7.4), 33 uM biotin, 17 uM pantothenate, 100 nM human insulin, 1 uM dexamethasone (DEX), 60 U/mL penicillin, 60 U/mL streptomycin, 25 ug/mL fungizone, 0.25 mM isobutylmethylxanthine (IBMX), and TZD (Exp. 1 & 2=BRL 49653; Exp. 3-5=Zen Bio's proprietary reagent). Thereafter, cultures were exposed to adipocyte medium consisting of 90% DME/Ham's F-10 (1:1,v/v), 15 mM HEPES (pH 7.4), 3% FBS (v/v), 33 uM biotin, 17 uM pantothenate, 100 nM human insulin, 1 uM DEX, 60 U/mL penicillin, 60 U/mL streptomycin, and 25 ug/mL fungizone. Adipocyte media was replaced every 3 d. After 10-12 d under these culturing conditions, approximately 35% of the cells exhibited morphology of mature adipocytes. After 18 day in culture, the majority of the cells contained visual lipid droplets.

Experimental Designs.

Experiment 1 was designed to determine optimal culturing conditions during differentiation of primary cultures of SV cells isolated from human adipose tissue. Specifically, the experiment was designed to determine how seeding density and thiazolidinedione (TZD; PPARγ-agonist BRL 49653) concentration influenced TG content (ug/10⁶cells). SV cells were seeded at increasing densities (2-, 3-, or 4×10⁴/cm²) in differentiation media containing 0, 1, or 10 uM TZD (BRL 49653) in this 3×3 factorial design. All cultures received the same adipocyte media after 3 d of differentiation. The cultures were harvested on day 11-12 of differentiation and TG content and cell number were measured. Another set of cultures was stained with Oil Red O and counterstained with Mayer's hematoxylin to assess cellular differentiation potential (n=3 per treatment combination).

In Experiment 2 the impact of linoleic acid and the trans-10, cis-12 isomer of CLA on TG accumulation in these cells was examined. SV cells were seeded at a density of 3×10⁴/cm² and continuously treated with either 10 uM linoleic acid, 100 uM linoleic acid, 10 uM trans-10, cis-12 CLA, or 100 uM trans-10, cis-12 CLA, and grown under our normal differentiation protocol. One set of control cultures contained only vehicle (BSA) and another contained vehicle plus TZD (BRL 49653). TG content and cell number were measured on day 11 of differentiation.

The objective of Experiment 3 was to evaluate the dose response of trans-10, cis-12 CLA, cis-9, trans-11 CLA, and linoleic acid on TG content of the cultures. SV cells were seeded at a density of 3×10⁴/cm² and continuously treated with increasing concentrations (1, 3, 10, or 30 uM) of either linoleic acid, cis-9, trans-11 CLA, or trans-10, cis-12 CLA. A set of control cultures contained only the vehicle (BSA) plus TZD (Zen Bio's proprietary agent). TG content and cell number were evaluated on day 11 of differentiation.

Experiment 4 was designed to determine if supplementing the cultures with linoleic acid could reverse the trans-10, cis-12-mediated reduction in TG content. SV cells were seeded at a density of 3×10⁴/cm² and continuously treated with either 10 uM trans-10, cis-12 CLA alone, 10 uM trans-10, cis-12 CLA plus linoleic acid at 10, 30, or 100 uM, or linoleic acid alone at 10, 30, or 100 uM. A set of control cultures contained vehicle (BSA) plus TZD (Zen Bio's proprietary agent). TG and cell number were assessed on day 11 of differentiation.

Experiments 5a and 5b were designed to determine if the trans-10, cis-12 CLA-mediated reduction in TG content was due to decreased lipogenesis and/or increased lipolysis. In Experiment 5a (lipogenesis), SV cells were seeded at a density of 3×10⁴/cm² and continuously treated with increasing concentrations (3, 10, or 30 uM) of either linoleic acid, cis-9, tran-11 CLA, or trans-10, cis-12 CLA. A set of control cultures received vehicle (BSA) plus TZD. All cultures received differentiation media (days 1-3), adipocyte media (days 4-9), and low-glucose (˜5 mM) adipocyte media (days 10-12) prior to measuring de novo lipogenesis. On day 12, ¹⁴C-labeled glucose incorporation into the lipid fraction of the cultures was measured for 2 h and, following lipid extraction, the radioactivity in the lipid fraction was determined by scintillation counting. Time course data (not shown) indicated a linear increase in radiolabeled glucose incorporation into lipid over 2 h.

In Experiment 5b, basal lipolysis was measured on day 18 of differentiation after the cultures had been treated with fatty acids for 5 h. Cultures were grown in basal media (e.g., adipocyte media lacking DEX and insulin) for 24 h before the measurement of lipolysis. Lipolysis was determined by measuring free and esterified glycerol release into the media following acute (5 h) treatment. A set of vehicle control cultures was treated with 1 uM isoproterenol to determine the lipolytic sensitivity of the cultures to a adrenergic agent known to activate adenylate cylase.

Treatment Specifications

Linoleic acid (Nu-check-prep, Elysian, Minn.; 99% pure), cis-9, trans-11 CLA (Matreya, Inc., Pleasant Garden, Pa.; 98% pure), and trans-10, cis-12 CLA (Matreya, Inc.; 98% pure) were complexed to fatty acid free albumin (1 mM BSA: 4 mM fatty acid), and added to post-confluent SV cultures at various concentrations, with exception to Experiment 5b in which all fatty acids were dissolved in DMSO. All cultures contained the same amount of vehicle (BSA). All cultures received differentiation media for days 1-3 and adipocyte media from day 4 onward unless otherwise indicated. Fresh fatty acids were added with each media change until the day of harvest. With the exception of Experiment 5b (lipolysis), all cultures were chronically treated with fatty acids (e.g., beginning on day 1 of the differentiation program) until their time of harvest during late stages of differentiation (days 10-18). All of the treatment combinations had a sample size of n=6 unless otherwise indicated.

Determination of Cell Number.

Adherent cells were harvested in 500 uL cell counting solution containing 0.01 M monobasic NaPO₄, 0.154 M NaCl, 25 mM EDTA, and 2%BSA. After gentle tritaration to deter cell clumping, cell number was determined using the Coulter Multi-Sizer IIE Counter (Coulter Electronics, Hialeah, Fla.).

Quantification of Triglyceride Content.

Cells were harvested in 500 uL cell counting solution and sonicated. Five percent (v/v) Triton X-100 was added to all lysates to ensure homogenous lipid distribution in all samples. Intracellular TG content was measured using a colorimetric assay that quantifies the glycerol content of the samples (GPO-Trinder #339-10, Sigma; St. Louis, Mo.). This assay involves the enzymatic hydrolysis of TG by lipases to free fatty acid and glycerol. The glycerol moiety, through a series of oxidation-reduction reactions, then associates with 3,5 dichloro-2-hydroxybenzene sulfonate and 4-aminoantipyrine to produce a red colored dye. The absorbency of this dye is directly proportional to the concentration of TG present in each lysate. Each sample was transferred to a 96 well plate, and the absorbency is quantified at 520 nm on a microtiter plate reader (Tecan-SLM, Research Triangle Park, N.C.). TG data are expressed as ug of TG per 10⁶ cells.

Lipid Staining.

The presence of intracellular lipid was visualized by staining cultures with Oil Red O as previously described for human SV cultures (McIntosh et al. (1999) Int. J. Obesity 23:595-602). Briefly, cell monolayers were washed twice with 1 mL Hank's Balanced Salt Solution (HBSS), and then fixed for 1 h in a 10% formalin solution (10% formalin, 4% calcium chloride, and deionized water) at 4° C. After fixation, cells were washed twice with deionized water and stained using a 0.3% Oil Red O in isopropanol for 15 min at room temperature. The cells were rinsed again with deionized water. The nuclei were then counterstained with Mayer's Hematoxylin (1 g/L) for 3 min, then rinsed a final time with deionized water for 3 min. Counterstaining allows for quantifying the percentage of cells that have undergone differentiation (e.g., total cell number per field/number of cells having appreciable amounts of Oil Red O stain). Photomicrographs were taken of the Oil Red O stained cells to provide visual indication of the degree of lipid accumulation in relation to nuclei.

De Novo Lipogenesis.

Incorporation of ¹⁴C-glucose into cellular lipid was determined on day 12 of differentiation in cultures chronically treated with fatty acids or vehicle. Following the addition of fatty acids and low glucose (˜5 mM) medium to the cultures on day 12, 1.0 μCi [U-¹⁴C]-glucose ([U-¹⁴C]-D-glucose; SA ˜250 mCi/mmol, ICN, Costa Mesa, Calif.)/mL medium was added to the cultures for 2 h. Our time course study (data not shown) indicated a linear increase in radiolabeled glucose incorporation into lipid over a 2 h period. After 2 h, media containing unincorporated ¹⁴C-glucose was immediately removed and the cultures were washed with 1 mL HBSS to remove unincorporated ¹⁴C-glucose. An additional 1 mL of HBSS was added and, after vigorous tritaration, cells were transferred to glass vials. Five milliliters of a chloroform:methanol (2:1) solution was added to each vial and they were vortexed for 1 min. All samples were then centrifuged for 5 min at 1000×g to further separate the hydrophobic and hydrophilic phases. The lower hydrophobic phase was removed from the tubes and dried under nitrogen at 40° C. Five milliliters of scintillation cocktail (Scinti Verse, Fisher Scientific, Norcross, Ga.) was added to each sample, and the ¹⁴C content was determined by liquid scintillation counting on a Beckman LS 6000 Scintillation Counter (Beckman Instruments, Palo Atlo, Calif.). To control for unincorporated ¹⁴C-glucose that may have accompanied the cultures into the lipid extraction vials, a set of cultures were exposed to ¹⁴C-glucose for 5 sec and subsequently washed, harvested, and fractionated. The radioactivity in the lipid fraction from these cultures was subtracted from the total counts. Cell numbers were determined from parallel treatment groups in separate culture dishes at the time of radioisotope addition to the medium. Therefore, mean ¹⁴C-glucose incorporation is expressed as cpm/10⁶ cells.

Lipolysis Assay.

On day 17 of differentiation, cultures were grown in basal adipocyte media (adipocyte media minus insulin and DEX). On day 18 of differentiation, cultures of mature adipocytes were washed and incubated in Krebs-Ringer buffer supplemented with ˜5 mM glucose and incubated for 5 h at 37° C. with the fatty acids treatments or 1.0 uM isoproterenol (positive control for lipolysis). All fatty acids were dissolved in DMSO (final concentration=0.1%). A set of vehicle controls contained 0.1% DMSO. One hundred microliters of conditioned media was removed from each well, and lipolytic rate determined by quantifying the amount of free glycerol and esterified glycerol in each sample using Sigma's triglyceride kit (GPO-Trinder, Sigma Chemical Co.).

Statistics.

Analyses of statistically significant differences between treatment means (e.g., main effects and their interactions) were conducted using two-way (e.g., Exp. 1=Seeding Density×BRL Concentration; Exp. 2-5=Treatment×Dose) analysis of variance (ANOVA) procedures and a commercially available software program (SUPERANOVA; Abacus Concepts, Berkeley, Calif.). Differences between treatment means were considered significant at P<0.05.

### Results

Experiment 1:

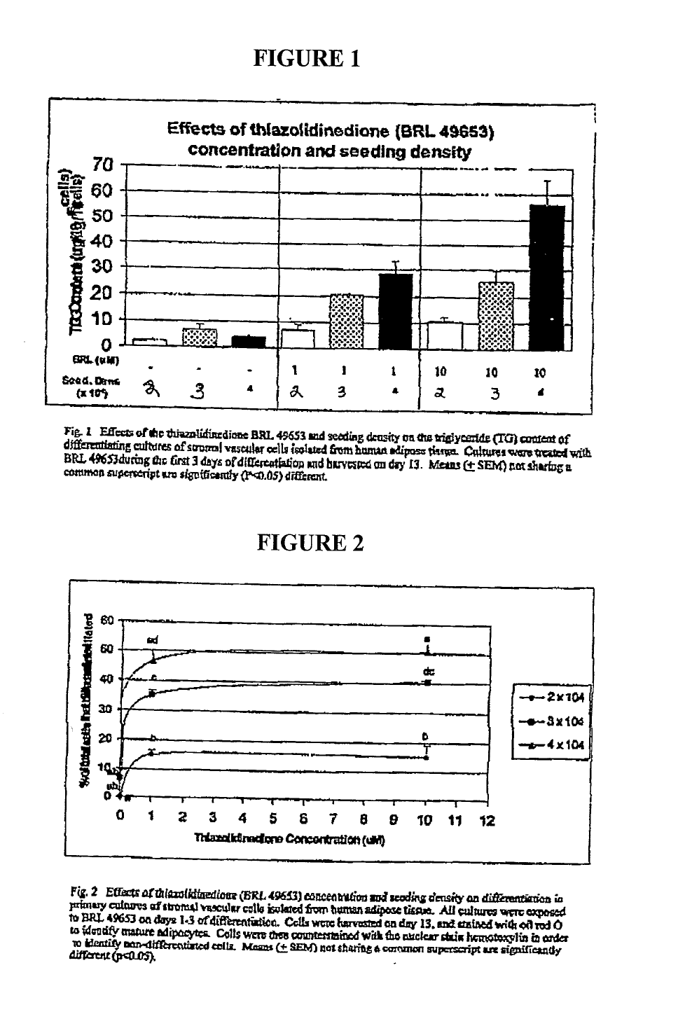

Increasing seeding density and TZD concentration increased the TG content (ug/10⁶ cells) of the cultures (FIG. 1A). The influence of increasing seeding density on TG content was greatest in the cultures containing either 1 or 10 uM TZD. This effect was greatest in cultures supplemented with 10 uM TZD, where doubling the seeding density increased the TG content approximately 5-fold. Data in FIG. 1B provides insight into how seeding density and TZD concentration influence the number of cells that phenotypically differentiate into adipocytes (e.g., accumulate visually-detectable lipid droplets). These data in FIG. 1B closely parallel the TG content data in FIG. 1, suggesting that the increase in TG content was due to an increase in the number of cells that have differentiated into adipocytes. The exception to this observation was the cultures seeded at the highest seeding density and TZD concentration. This treatment group had almost twice as much TG content as compared to the group seeded at the same density (4×10⁴) and supplemented with 1 uM TZD, but had almost the same percentage of cells that differentiated (47 vs. 50%). This suggests that the increase in TZD concentration from 1 to 10 uM increased adipocytes size rather than adipocyte number.

Experiment 2.

Cultures treated with 10 uM trans-10, cis-12 CLA had 76% less TG (ug/10⁶ cells) than vehicle control cultures that were supplemented with TZD (FIG. 2). Interestingly, cultures treated with 100 uM trans-10, cis-12 CLA were not significantly different than cultures treated with 10 uM linoleic acid or the TZD treated vehicle controls. Cultures treated with 100 uM linoleic acid had the highest TG content.

Experiment 3.

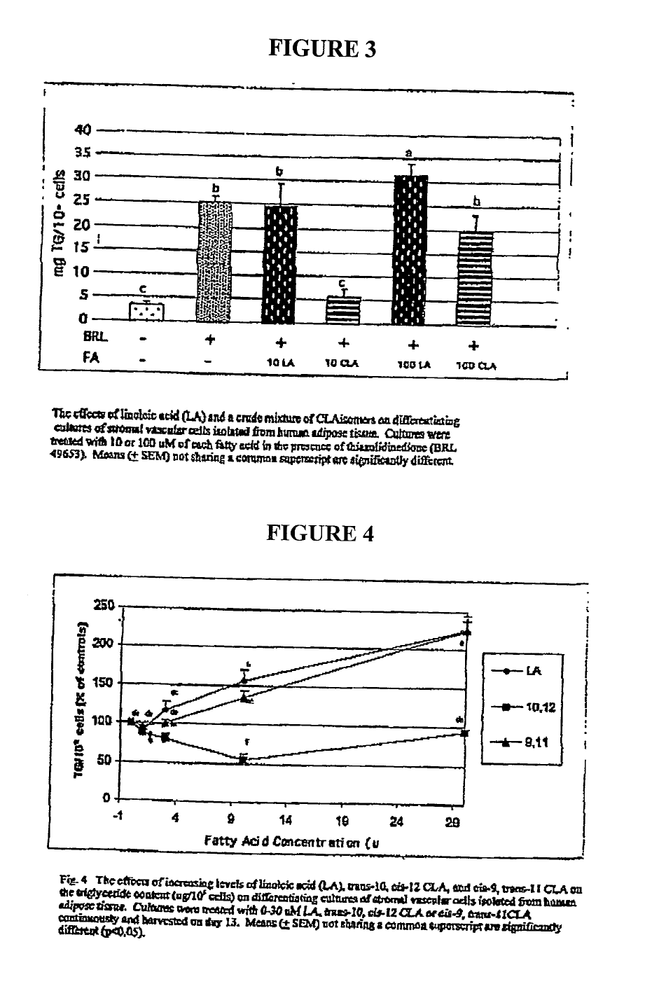

The TG content of the cultures increased in a dose dependent fashion as the level of linoleic acid and cis-9, trans-11 CLA increased (FIG. 3). In contrast, as the level of trans-10, cis-12 increased from 1-10 uM, the TG content of decreased. However, TG content of cultures treated with 30 uM trans-10, cis 12 CLA were not significantly different than the TZD treated vehicle controls.

Experiment 4.

Cultures treated with 10 uM trans-10, cis-12 CLA alone had approximately 60% less TG content compared to the TZD treated vehicle controls (FIG. 4). Interestingly, when 10 uM trans-10, cis-12 CLA-treated cultures were supplemented with 10, 30, or 100 uM linoleic acid, they had 26, 55, and 64% more TG content, respectively, than those cultures treated with 10 uM trans-10, cis-12 CLA alone. In fact, the trans-10, cis-12 CLA-treated cultures supplemented with 100 uM linoleic acid had a TG content similar to the TZD treated BSA controls, suggesting linoleic acid supplementation reverses the TG lowering effect of CLA.

Experiment 5a.

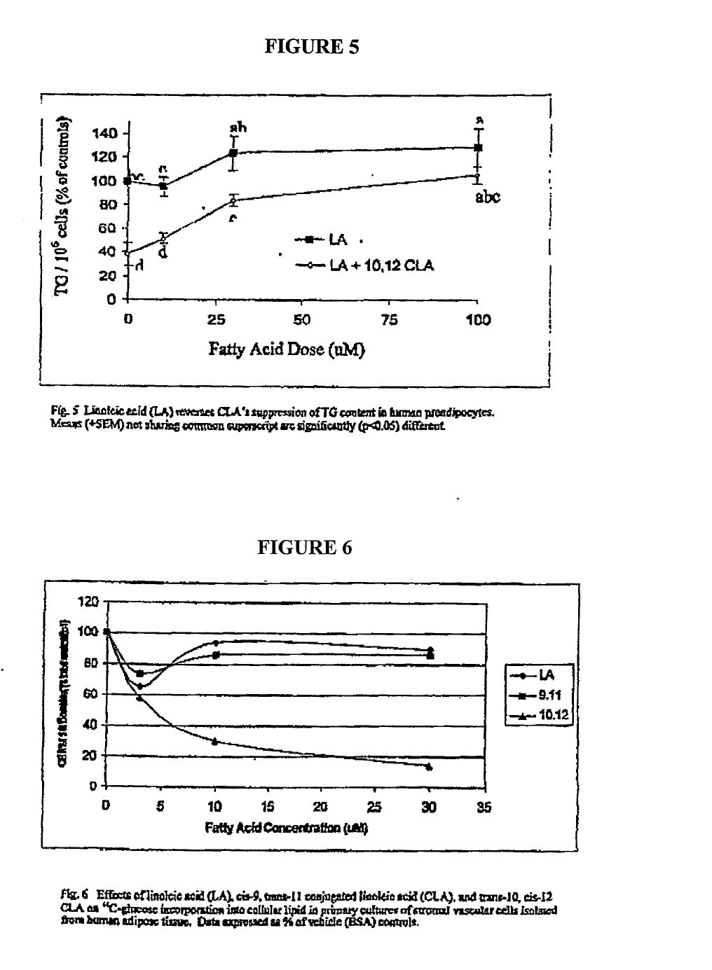

Incorporation of ¹⁴C-glucose into cellular lipid per 10⁶ cells decreased as the level of trans-10, cis-12 CLA increased in the cultures (FIG. 5). Cultures treated with 30 uM trans-10, cis-12 CLA had 80% less ¹⁴C-glucose incorporated into cellular lipid compared to the TZD treated vehicle controls. In contrast, neither linoleic acid nor trans-9, cis-11 CLA influenced de novo lipogenesis.

Experiment 5b.

Isoproterenol stimulated lipolysis in cultures of adipocytes from abdominal and thigh approximately 2.5- and 1.5-fold, respectively. In contrast, lipolysis was not altered by any of the acute fatty acid treatments in cultures of abdominal adipocytes compared to the DMSO controls. In cultures of thigh adipocytes, although all fatty acid treatments slightly stimulated lipolysis, there were not significant differences in lipolysis between the types or doses of fatty acids.

### Discussion

It was confirmed that trans-10, cis-12 CLA is the isomer of CLA that is responsible for the TG-lowering effects of a commercially available crude mixture of CLA isomers using 3T3-L1 preadipocytes as the cell model. However, the effects of trans-10, cis-12 CLA are dependent on dose, duration, and time period of treatment, as treatment throughout the first 6 d of differentiation was more effective than either treatment during the first 3 d or the last 3 d of differentiation. Our results substantiate the reports of previous research demonstrating that trans-10, cis-12 CLA is the antiadipogenic isomer of CLA. In vivo, ICR mice consuming 0.25% trans-10, cis-12 enriched CLA had lower body fat percentages than controls or mice fed 0.25% cis-9, trans-11 enriched CLA (Park et al. 1999b). Furthermore, Baumgard et al. (2000) Am. J. Physiol. 278:R179-R184 found that only the trans-10, cis-12 isomer of CLA reduced milk fat percentage and yield in Holstein cows. In vitro, Park et al. (1999b) showed that 3T3-L1 preadipoctyes treated for 48h with trans-10, cis-12 CLA contained less intracellular TG and glycerol than cis-9, trans-11 CLA-treated cultures. More recently, Choi et al. (2000) J. Nutr. 130:1920-1924, found that trans-10, cis-12 CLA inhibited the production of stearoyl-CoA desaturase-1 (SCD-1) without reducing PPARγ2 or aP2 mRNA levels in 3T3-L1 preadipocytes.

To our knowledge, our results are the first to show that the impact of trans-10, cis-12 CLA on 3T3-L1 preadipocytes depends on the time period of treatment. Data in FIG. 2 clearly show that treatment during the entire period of differentiation reduced TG content to a greater extent than treatment during the last 3 d of differentiation. However, trans-10, cis-12 CLA treatment during only the first 3 d of differentiation was not sufficient to lower TG concentrations. These results are similar to in vivo studies of Park et al. (1999a) who found that whole body fat percentages of ICR mice fed 0.25% CLA for 4 wk began to approach the levels of control mice once CLA was removed from the diets. However, the CLA-fed mice still had lower body fat percentages than those of control mice. Furthermore, Park et al. (1999b) showed that 3T3-L1 preadipocytes treated with mixed isomers of CLA for 48 h on day 4 of differentiation had lower TG levels than LA controls, results which correspond to our effects seen with treatment during the last 3 d of differentiation.

In an attempt to elicit a mechanism through which trans-10, cis-12 CLA inhibits TG accumulation, the expression of PPARγ2 and aP2 protein was assessed. In contrast to the hypothesis that trans-10, cis-12 CLA reduces TG content by reducing the expression of PPARγ2, it was determined that the trans-10, cis-12 isomer of CLA increased PPARγ2 expression on day 2 of differentiation compared to BSA controls, while having no effect on PPARγ2 protein expression on day 4. aP2 protein levels were unaffected by either 2 or 4d of CLA treatment. Interestingly, LA treatment reduced PPARγ2 protein expression. In support of this unexpected result, linoleic acid has previously been suggested to inhibit preadipocyte differentiation and SCD-1 gene expression (Casimir and Ntambi (1996) Differerentiation 60:203-210). Furthermore, it was consistently found that LA treatment increased cell number (data not shown) which would correlate with a suppression of PPARγ2 expression to allow clonal expansion during the early phase of differentiation (Ailhaud et al. (1992) Ann. Rev. Nutr. 12:207-233, MacDougald and Lane (1995) Ann. Rev. Biochem. 64:345-373).

This is the first report of the impact of trans-10, cis-12 CLA treatment on PPARγ2 protein levels in 3T3-L1 preadipocytes. Brodie et al. (1999) found that 50 uM of a crude mixture of CLA isomers reduced PPARγ mRNA expression in 3T3-L1 preadipocytes treated for 2-7 d. However, these researchers used a probe which was a general probe for PPARγ, not one that was specific for PPARγ2-the isoform that controls adipogenesis. Furthermore, the combination of CLA isomers may have differential effects on PPARγexpression. In contrast to Brodie et al.'s results, Choi et al. (2000) reported that although a crude mixture of CLA isomers reduced PPARγ2 mRNA expression, 45 uM trans-10, cis 12 CLA did not affect PPARγ2 mRNA levels. Furthermore, Houseknecht et al. (1999) found that 100-200 uM of a crude mixture of CLA isomers activated the expression of PPARγ in CV-1 cells transiently transfected with a human PPARγ reporter gene construct.

It was determined that trans-10, cis-12 CLA did not affect aP2 protein expression on either day 2 or 4 of 3T3-L1 preadipocyte differentiation which correlates with the results of Choi et al.'s mRNA data. In contrast, in vivo, Houseknecht et al. (2000) found that 1.5% (w/w) of a crude mixture of CLA isomers increased aP2 mRNA levels in prediabetic ZDF mice. However, the effect of the individual CLA isomers on aP2 expression is unknown. Furthermore, these researchers used epididymal adipose tissue that contains a mixture of stromal vascular cells, including preadipocytes and adipocytes in various stages of differentiation, making the interpretation of these results unclear.

For the lipid-lowering effects of CLA to be physiologically relevant, CLA must incorporate into cellular lipids or alter lipid composition. To this end, it was determined that both trans-10, cis-12 CLA and cis-9, trans-11 CLA incorporated into the neutral and phospholipid fractions; however, the cis-9, trans-11 CLA isomer was 1-2 fold more abundant than the trans-10, cis-12 isomer. In agreement with these data, Comb White Leghorn laying hens fed mixed isomers of CLA had higher levels of cis-9, trans-11 CLA than trans-10, cis-12 CLA in their egg yolks (Jones et al. 2000). In addition, Albino rats fed 0.5, 1.0, or 1.5% (w/w) of a crude mixture of CLA isomers for 60 d had almost twice the amount of cis-9, trans-11 CLA in their adipose tissue as trans-10, cis-12 CLA (Szymczyk et al. 2000). In contrast, Sprague-Dawley rats fed 0.25-0.5 (g/100 g diet) of a crude mixture of CLA isomers for 5 weeks had similar amounts of cis-9, trans-11 and trans-10, cis-12 CLA in their retroperitoneal fat pads compared to controls (Azain et al. 2000).

CLA's antiadipogenic actions have been proposed to be due to an inhibited elongation and/or desaturation of unsaturated fatty acids such as palmitic acid and stearic acid into polyunsaturated fatty acids (Choi et al. 2000). In support of this hypothesis, it was found that 50 uM trans-10, cis-12 CLA treated cultures had lower amounts of palmitoleic acid (16:1) (in the neutral lipid fraction) and cis-11 oleic acid (18:1) (in both the neutral and polar lipid fractions) compared to BSA controls. However, trans-10, cis-12 CLA increased the amount of cis-9 oleic acid as well as the level of linoleic acid (18:2). These results differ from those of Satory and Smith 1999 who found that 3T3-L1 preadipocytes cultured with 5-10 mg/L of mixed isomers of CLA had increased amounts (g/100 total fatty acid) of palmitic acid (16:0) and palmitoleic acid (16:1) and decreased stearic acid (18:0), and oleic acid (18:1) concentrations. However, these researchers treated their preadipocytes with the crude mixture of CLA isomers, dissolved their fatty acids in ethanol as the delivery vehicle, evaluated a combination of neutral and polar lipids, and used lower amounts of CLA, all of which could account for the differences in our results. More recently, Choi et al. (2000) showed that 3T3-L1 preadipocytes treated with 45 uM trans-10, cis-12 CLA lower levels of both 16:1 and 18:1 in their cellular lipids, similar to our results in neutral lipids where TG is stored. Finally, Δ-6 desaturation of linoleic acid in rat hepatic microsomes was decreased in the presence of both cis-9, trans-11 and trans-10, cis-12 CLA (Bretillon et al. 1999). However, only the trans-10, cis-12 isomer of CLA inhibited Δ-9 desaturation of stearic acid.

Numerous in vivo studies have also shown that CLA treatment alters the production and/or metabolism of long chain fatty acids, especially the production of 16:1 and 18:1 from 16:0 and 18:0, respectively. For example, Azain et al. (2000) J. Nutr. 130:1548-1554 found that Sprague-Dawley rats fed 0.5% mixed isomers of CLA for 7 or 49 d had lower levels of 16:1 and 18:1, along with higher levels of 18:2 in their adipose tissue. In addition, weanling lambs fed 0.33 g/d of mixed isomers of CLA for 101 d had decreased levels of 18:0, 18:1, and 18:2 in their adipose tissues (Mir et al. 2000). In contrast, Bee (2000) J. Nutr. 130:2292-2298, showed that the backfat of pregnant sows had higher levels of 16:0, while concentrations of n-9 18:1, and 18:2 decreased. Similarly, Szymczyk et al. (2000) J. Sci. Food Agric. 80:1553-1558, found that levels of cis-9 18:1 increased while 16:1, 18:2, and 20:4 concentrations decreased in rats fed 0.25-0.5 g/100 g diet of mixed isomers of CLA for 5 weeks. Therefore, it is still unclear what effect CLA has on the fatty acid profile of adipose tissue and adipocytes in culture, especially since few studies have been conducted with the trans-10, cis-12 isomer of CLA. However, there is some evidence to suggest that trans-10, cis-12 CLA may interfere with the production of certain monounsaturated fatty acids such as 16:1 and 18:1.

CLA treatment has also been theorized to inhibit the production of adipogenic fatty acids such as arachidonic acid (AA) and its subsequent eicosanoid metabolites. A reduction in AA and other adipogenic fatty acids may decrease TG esterification, conversion into phospholipids that are critical for cellular metabolism, and/or synthesis into lipid second messengers, such as PGJ₂, that may regulate adipogenesis. In contrast to this theory, our data revealed a dose-dependent increase in AA in the phospholipid fraction as the level of trans-10, cis-12 increased in the culture. Our results differ from the in vivo studies of Azain et al. (2000) J. Nutr. 130:1548-1554, Szymczyk et al. (2000), and Bee (2000) who found that the CLA-fed rats and sows had lower levels of 20:4 in their adipose tissue. However, it is difficult to compare our results with these in vitro studies as the above researchers used mixed isomers of CLA and did not separate the neutral and polar lipids prior to analysis.

Our results also conflict with those of Satory and Smith (1999) J. Nutr. 129:92-97who found that 3T3-L 1 preadipocytes cultured with 5-10 mg/L of a crude mixture of CLA isomers had lower levels of AA. However, Choi et al. (2000) found that while mixed CLA treatment had no effect on 20:4 concentrations in 3T3-L1 preadipocytes, treatment with trans-10, cis-12 CLA increased 20:4 concentrations. Finally, Liu and Belury (1998) Cancer Lett. 127:15-22, found that keratinocytes cultured with 5 or 16 ug/mL of mixed CLA isomers for 12 h had lower AA concentrations than LA-treated cells; however there was no difference in CLA-treated AA concentrations compared to control cultures. Thus, our results, along with those of Choi et al. (2000) and Liu and Belury (1998), dispute the suggestion that CLA's antiadipogenic actions may be the result of an inhibition of adipogenic fatty acid production. However, since the production of eicosanoid metabolites and lipid second messengers such as PGJ₂ that may impact adipogenesis was not assessed, it is still possible that trans-10, cis-12 CLA may be inhibiting TG production through these pathways.

Lastly, in a fourth set of experiments, the impact of LA supplementation on trans-10, cis-12 CLA-treated cultures' TG content, morphology, and adipogenic protein expression was examined. This is believed to be the first report that concurrent treatment with linoleic acid (18-2, n-6) was able to prevent the TG-lowering effects of trans-10, cis-12 CLA. Furthermore, the increase was dose-dependent and almost reached the level of LA-only supplemented cultures. These results may indicate that trans-10, cis-12 CLA's TG-lowering effects can be overcome by supplementation with PUFAs. Further research to determine if other fatty acids have differential rescue effects dependent on structure are in progress.

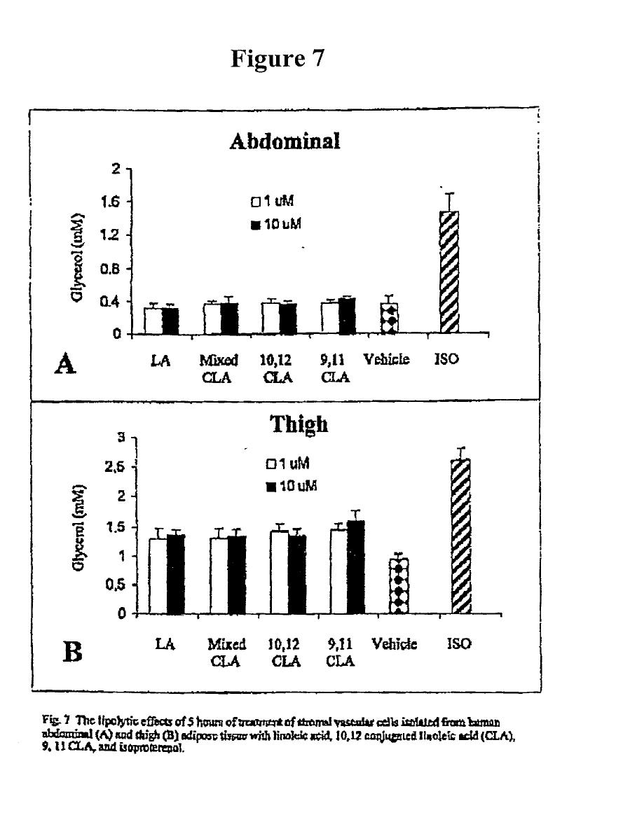

Treatment with both mixed CLA isomers and trans-10, cis-12 CLA induced biochemical (i.e., nuclear condensation and increased percentage of cells in the sub-G₁ phase) and morphological changes (i.e., rounding and membrane blebbing)-changes that are characteristic of apoptosis (Evans et al. (2000)). In the current research, supplementation of trans-10, cis-12 CLA-treated cultures with LA inhibited the CLA-induced morphological changes in a dose-dependent manner. In agreement with these data, CLA-treated cultures supplemented with LA have greater TG content (FIG. 7). A number of studies have also shown that CLA is capable of inducing apoptosis. For example, cells in the adipose tissue of C57BL/6J mice fed 1% (w/w) of mixed isomers of CLA underwent apoptosis (Tsuboyama-Kasaoka et al. 2000). Additional studies in primary rat mammary cells (Ip et al. 1999) as well as NMU mammary cells (Ip et al. 2000) have also demonstrated that CLA induces apoptosis. The mechanism through which LA prevents trans-10, cis-12 CLA's induction of apoptosis is unclear; however, one possibility is that CLA treatment may induce the expression of TNFα, a known inducer of apoptosis (Pariza et al. (2000) PSEBM 223:8-13; Tsuboyama-Kasaoka et al. (2000) Diabetes 49:1534-1542).

Finally, PPARγ2 and aP2 protein expression after 6 d of supplementation with trans-10, cis-12 CLA and LA were examined. In these experiments, it was found that LA treatment decreased both PPARγ2 and aP2 protein expression. This reduction in PPARγ2 was similar to that seen in the earlier studies of PPARγ2 expression on day 2 of differentiation. In addition, the expression of PPARγ2 and aP2 proteins decreased with trans-10, cis-12 CLA after 6 d of treatment-a result which was not seen after 2 or 4 d of treatment in experiment 2. Similar to our results, Brodie et al. (1999) found that both LA and mixed CLA reduced PPARγ2 mRNA levels on day 7 of differentiation. Surprisingly, concurrent LA and trans-10, cis-12 CLA treatment for 6 d increased PPARγ2 protein levels compared to trans-10, cis-12 CLA treatment alone, an effect which may explain some of LA's ability to reverse CLA's antilipogenic effects. However, the exact mechanism through which LA is able to reverse the decrease in PPARγ2 expression and TG-lowering effect of CLA remains to be determined.

In conclusion, it was found that trans-10, cis-12 CLA is the TG-lowering isomer of CLA in 3T3-L1 preadipocytes. Furthermore, trans-10, cis-12's effects are time and dose-dependent and do not appear to depend directly on a reduction in PPARγ2 or ap2 protein expression. This work also has led to the discovery that trans-10, cis-12 CLA decreased the production of certain monounsaturated fatty acids such as 16:1 and cis-11 oleic acid, while increasing linoleic acid and arachidonic acid levels. Finally, supplementation with linoleic acid was able to prevent some, but not all, of trans-10, cis-12 CLA's TG-lowering effects. Moreover, these effects may be the result of an inhibition of apoptosis and/or an induction of adipogenic protein expression. Future research is needed to discover the mechanism through which trans-10, cis-12 decreases TG content.

We have previously shown that both a commercially available mixture of conjugated linoleic acid (CLA) isomers and the trans-10, cis-12 isomer of CLA reduced the triglyceride (TG) content and induced apoptosis in differentiating cultures of 3T3-L1 preadipocytes. However, the influence of CLA isomers on the differentiation of human (pre)adipocytes is unknown. Therefore, we conducted a series of studies using primary cultures of stromal vascular (SV) cells isolated from human adipose tissue to determine: 1) the influence of seeding density and thiazolidinedione (TZD) concentration on TG content; 2) whether linoleic acid or trans-10, cis-12 CLA altered TG content; 3) the dose response of cis-9, trans-11 CLA vs. trans-10, cis-12 CLA on TG content; 4) whether linoleic acid supplementation could rescue the TG content of CLA-treated cultures; and 5) if the trans-10, cis-12 mediated reduction in cellular TG was due decreased de novo lipogenesis and/or increased lipolysis. In Experiment 1, the TG content (ug/10⁶ cells) increased as both seeding density and TZD concentration increased. For example, cultures seeded at 4×10⁴ cells/cm² and supplemented with 10 uM BRL 49653 had 10-fold greater TG content than similarly seeded cultures without BRL 49653. In Experiment 2, chronic treatment with 10 uM trans-10, cis-12 CLA decreased the TG content compared to vehicle (BSA) and linoleic acid-treated controls. In Experiment 3, TG content decreased as the level of trans-10, cis-12 CLA increased from 1-10 uM, whereas the TG content increased with increasing concentrations of linoleic acid and cis-9, trans-10 CLA. In Experiment 4, linoleic acid supplementation restored the TG content of cultures treated with trans-10, cis-12 CLA compared to cultures treated with CLA alone, suggesting CLA's attenuation of TG content is reversible. In Experiment 5, de novo lipogenesis decreased with increasing levels of trans-10, cis-12 CLA, whereas neither isomer of CLA acutely impacted lipolysis. These data suggest that the recently reported antiobesity actions of a supplement containing a crude mixture of CLA isomers given to humans made be specifically due to the trans-10, cis-12 isomer, which decreases de novo lipogenesis in vitro.

More recently, it was demonstrated that a commercially available mixture of CLA isomers and the trans-10, cis-12 isomer lowered the TG content and induced apoptosis in cultures of 3T3-L1 preadipocytes (Evans et al. 2000). A recent study found that trans-10, cis-12 CLA reduced steroyl-CoA desaturase (SCD-1) activity and mRNA levels without affecting PPARγ or aP2 mRNA, suggesting that CLA may be interfering with the desaturation of long-chain fatty acids and their subsequent esterification into TG (Choi et al. 2000).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for reducing the signs of cellulite in a patient, the method comprising applying onto the skin of said patent, a composition comprising:
(a) 10-trans, 12-cis conjugated linoleic acid; and
(b) a cosmetically acceptable carrier.

2. A method according to claim 1, wherein the concentration of 10-trans, 12-cis conjugated linoleic acid is from about 0.1% to about 10%.

3. A method according to claim 1, wherein the composition further comprises an additional active selected from the group consisting of phosphodiesterase inhibitors, oleosoluble vegetable extracts, herbal extracts, botanical extracts and mixtures thereof.

4. A method according to claim 3, wherein the additional active is a phosphodiesterase inhibitor selected from the group consisting of theophylline, caffeine, theobromine, salts thereof and mixtures thereof.

5. A method according to claim 1, wherein the composition further comprises an additional skin active selected from the group consisting of hydroxy acids, desquamatory agents, sunscreens, anti-oxidants, retinoids and mixtures thereof.

6. A method according to claim 5, wherein the hydroxy acid is salicylic acid; the desquamatory agent is selected from the group consisting of zwitterionic surfactants and mixtures thereof; the sun-block is selected from the group consisting of zinc oxide, titanium dioxide and mixtures thereof, the sunscreen is selected from the group consisting of 2-ethylhexyl-p-methoxycinnamate, 4,4′-t-butyl methoxydibenzoyl-methane, phenyl benzimidazole sulfonic acid, octocrylene and mixtures thereof; the anti-oxidant is selected from the group consisting of tocopherol, esters thereof and mixtures thereof; and the retinoid is selected from the group consisting of retinol, retinyl acetate, retinyl propionate, and mixtures thereof.

7. A method according to claim 1, wherein the skin care composition is contained within a patch or is applied to the skin and covered by a patch.

8. A composition comprising 10-trans, 12-cis conjugated linoleic acid and a pharmaceutically acceptable carrier.

9. The composition of claim 8, wherein the concentration of 10-trans, 12-cis conjugated linoleic acid is at least about 0.1%

10. A composition comprising 10-trans, 12-cis conjugated linoleic acid and a cosmetically acceptable carrier.

11. A composition according to claim 9, wherein the concentration of 10-trans, 12-cis conjugated linoleic acid is from about 0.1% to about 10%.

12. A composition according to claim 9, wherein the composition further comprises an additional active selected from the group consisting of phosphodiesterase inhibitors, oleosoluble vegetable extracts, herbal extracts, botanical extracts and mixtures thereof.

13. A composition according to claim 12, wherein the additional active is a phosphodiesterase inhibitor selected from the group consisting of theophylline, caffeine, theobromine, salts thereof and mixtures thereof.

14. A composition according to claim 9, wherein the composition further comprises an additional skin active selected from the group consisting of hydroxy acids, desquamatory agents, sunscreens, anti-oxidants, retinoids and mixtures thereof.

15. A composition according to claim 14, wherein the hydroxy acid is salicylic acid; the desquamatory agent is selected from the group consisting of zwitterionic surfactants and mixtures thereof; the sun-block is selected from the group consisting of zinc oxide, titanium dioxide and mixtures thereof; the sunscreen is selected from the group consisting of 2-ethylhexyl-p-methoxycinnamate, 4,4′-t-butyl methoxydibenzoyl-methane, phenyl benzimidazole sulfonic acid, octocrylene and mixtures thereof; the anti-oxidant is selected from the group consisting of tocopherol, esters thereof and mixtures thereof; and the retinoid is selected from the group consisting of retinol, retinyl acetate, retinyl propionate, and mixtures thereof.
